(19) United States
(12) Patent Application Publication
(10) Pub. No.: US 2012/0265076 A1
(43) Pub. Date: Oct. 18, 2012

(54) MICROFABRICATED CARDIAC SENSOR WITH TACTILE FEEDBACK AND METHOD AND APPARATUS FOR CALIBRATING THE SAME USING A PLURALITY OF SIGNALS

(76) Inventor: Stuart O. Schecter, Great Neck, NY (US)

(21) Appl. No.: 13/337,807

(22) Filed: Dec. 27, 2011

Related U.S. Application Data
(63) Continuation of application No. 12/245,058, filed on Oct. 3, 2008, now abandoned.
Application No. 12/245,058 is a continuation-in-part of application No. 11/334,935, filed on Jan. 19, 2006, now abandoned.
(60) Provisional application No. 60/660,101, filed on Mar. 9, 2005.

(51) Int. Cl. A61B 5/02; A61N 1/365; A61B 8/02; A61N 1/05; A61B 18/00; A61B 6/00; A61B 8/06; B82Y 5/00

(52) U.S. Cl. 600/455; 600/407; 607/17; 600/508; 600/454; 606/129; 606/41; 977/904; 977/742

(57) ABSTRACT

The cross-correlation of corresponding signals facilitates the development of sensor nanotechnologies including a catheter for performing ablation of cardiac arrhythmias and a biocompatible electrical interface with monitoring capabilities. Cross-correlation of data acquired with differing techniques enables system calibration and design, as well as, validation of the data acquired with next generation sensors. In a preferred mode of the invention, novel cardiac nanosensors enable an operator to differentiate one individual patient's cardiac tissue mechanical properties from others by using a sense of touch much as clinicians today use auditory cues with a stethoscope.

### RELATED APPLICATION

This application is a continuation of application Ser. No. 12/245,058 filed Oct. 3, 2008, which in turn is a continuation-in-part of application Ser. No. 11/334,935 filed Jan. 19, 2006, now abandoned, which claims the benefit of U.S. Provisional Application No. 60/660,101 filed Mar. 9, 2005 and U.S. Provisional Application No. 60/647,102 filed Jan. 26, 2005, each of which is hereby fully incorporated herein by reference.

### FIELD OF THE INVENTION

This invention pertains to a method for design of novel cardiac nanosensors in part by using data collected with multiple conventional apparatuses for calibration purposes. Comparisons of analogous data sets acquired with standard diagnostic equipment to that acquired with innovative rmicrofabricated cardiac sensors enables calibration of the latter and facilitates the analysis and interpretation of the newly acquired data. Open connectivity between multiple apparatuses and microfabricated sensors expedites the data collection process advancing our insights into the clinical relevance of newly defined sensor metrics and enables manufacturing of a haptic control system with tactile feedback.

### BACKGROUND OF THE INVENTION

Open connectivity between multiple diagnostic and therapeutic apparatuses is improving data collection, data analysis, and electronic medical record keeping. Data transfer using wireless telemetry between implanted cardiac devices and office based processing centers is becoming more widespread (e.g. Medtronic's Carelink, Boston Scientific's Latitude). Advances in the design of implanted cardiac sensors/transducers within permanently or temporarily inserted devices (heretofore referred as intrinsic sensors or signals) will enable acquisition of valuable data that traditionally is acquired from conventional diagnostic equipment (heretofore referred as extrinsic sensors or signals) such as MRI, CT scan, echocardiograms, cardiac navigational systems and nuclear imaging equipment. Such acquired data can be wirelessly uploaded into processing centers for storage, analysis and cross-correlation to data acquired with novel sensor technology.

Commonly used cardiac monitoring devices, such as echocardiography equipment, derive indices or metrics of cardiac function based on large volumes of data in various sub-groups of patients. Extensive amounts of research and data collection over many years has enabled extrinsically acquired cardiac data to have clinically applicability (e.g. guide treatment and yield prognosis). Open connectivity is enabling the development of new technologies based on a composite of collected data. In patent application 20040176679 and U.S. Pat. No. 743,333, Murphy et al. describe the design of cardiac instruments using computerized image data to create a pattern of at least one portion of an instrument for performing cardiac procedures. In patent application 20040153128, Sureshi, M and Dalton, J describe a method and system for image processing and contour assessment using wireless communication. In patent application 20050059876, Krishnan, S et al. implement medical records as part of an automated assessment of myocardial function using wall motion analysis methods. These prior art relate to imaging cardiac tissue to determine viability and direct cardiac interventions.

In this invention, novel microfabricated nanosensors are developed to provide anatomic and functional information along with tactile feedback to an operator. The clinical applicability of novel microfabricated sensors is realized in short order as open connectivity enables cross-correlation of collected data with conventional extrinsic modalities expediting comparisons of data between patients in various subgroups.

Incorporation of more advanced intra-cardiac sensor technology capable of cardiac monitoring at the level of conventional extra-cardiac apparatuses will require transducer miniaturization. Application of advances in nanotechnology facilitate such an endeavor. In this vein, calibration and standardization of the acquired data will become even more important.

Piezoelectric sensors or accelerometers are under development and hold promise to acquire intra-cardiac data representative of myocardial wall motion. Accelerometers that may be placed within electrode leads can be positioned juxtaposed to ventricular wall locations, such as the left ventricle free wall, right ventricle free wall, and the anterior/septal/lateral wall or intra-cardiac (e.g. endovascular). The accelerometers produce signals in response to the motion of the ventricular wall locations that relate to mechanical tissue characteristics during the cardiac cycle. An example of how this technology can be applied to programming of timing intervals within an implanted CRM device can be found in U.S. Pat. No. 7,121,289 by Yinghong Yu. Whereas existing sensor designs may be useful for comparing different tissue segments (e.g. right and left ventricular timing in a resynchronization device) these sensors fall short of being used for comparisons between patients as they are not calibrated from a physiologic standpoint and they are not standardized in different patient subgroups (i.e. according to level of pathology).

Navigational systems currently used for performing ablation of cardiac arrhythmias assist in three-dimensional intra-cardiac catheter positioning by merging computerized, anatomic three-dimensional displays of the heart. Extrinsic (i.e. externally applied) systems used to navigate about the heart include CARTO (Biosense-Webster), NavX (Endocardial Solutions, St. Paul, Minn., USA), LocaLisa (Medtronic, Minneapolis, Minn., USA). These technologies implement non-fluoroscopic methods including magnetic fields, externally applied electrical fields or ultrasound-distance mapping and merge these data with radiographic data (e.g. CT scans). By way of example, LocaLisa and NavX implement an externally applied electrical field (e.g. injected currents) detectable via standard catheter electrodes for real-time three dimensional localization of intra-cardiac catheters based on voltage drops over short distances. Localization accuracy of such systems is less than 2 mm. The details of such technology are known by those experienced in the art and are readily available in the scientific literature (Wittkampf FHM et al. LocaLisa, New Technique for Real Time 2 Dimensional Localization of Regular Intracardiac Electrodes. Circulation 1999; 99: 1312-1317). Other Three-Dimensional Mapping Systems include EnSite, RPM Mapping Systems, ESI Noncontact Mapping (Packer DL, Three-Dimensional Mapping of Intervential Electrophysiology: Techniques and Technology. Journal of Cardiovascular Electrophysiology 2005; Vol 16, No. 10, 1110-1117).

Most of the systems in use implement electric or electromagnetic fields. The ESI Noncontact Mapping implements a 64 electrode mesh mounted on the outside surface of a 18.times.40 mm balloon using 5.6 kHz currents driven between the rings on the mesh catheter and ablation catheter tip which is located in 3D space by sensing resulting potentials on the mesh electrodes. Thousands of calculated virtual electrograms, reflecting voltage transients from the endocardium are created using an inverse solution to the Laplace equation for intra-cardiac mapping. The RPM Mapping System (Real-time Position Management System) implements ultrasound-distance ranging with catheters positioned in the right ventricle, coronary sinus and a roving ablation catheter all of which transmit and receive ultrasound signals. The distance between catheters is based on calculations from the velocity of sound transmission in the heart and determination of the time between transmission and reception (Packer DL: Evolution and mapping and anatomic imaging of cardiac arrhythmias. J Cardiovasc Electrophysiol 2004; 15: 839-854).

In this invention, microfabricated novel sensor technologies are constructed in part based on data acquired with extra-cardiac apparatuses such as cardiac navigational systems Communication between the various apparatuses help calibrate the newly designed sensors facilitating our understanding of the anatomic and mechanical data that in turn provide tactile feedback to the operator. The newly designed sensors provide anatomic and mechanical information obviating the need for conventional extra-cardiac technologies. Thus, the extrinsic systems facilitate the manufacturing of intrinsic systems enabling them to replace their extrinsic predecessors in patients undergoing invasive cardiac procedures and in patients permanently implanted with cardiac devices.

### REFERENCES

The following references provide background information for the present application and illustrate the state of the art. All these references are incorporated by reference.
U.S. Pat. Nos. 6,804,559, 6,795,732, 6,792,308, 6,816,301, 6,572,560, 6,070,100, 6,725,091, 6,628,988, 6,740,033, 5,971,931, 5,833,623, 6,826,509, 6,805,667, 6,574,511, 6,418,346, 5,549,650, 6,077,236, 5,389,865, 5,769,640, 5,628,777, 5,693,074, 6,906,700, 6,780,183, 6,203,432, 6,641,480, 7,139,621, 7,121,289, 743,333
Published US patent applications: 20040176810, 20030083702, 20020026103, 20040111127, 20020072784, 20030216620, 20040167587, 20050182447, 20050043895, 20040186465, 200050241026, 20040176679, 20040153128, 20050059876

References in peer-reviewed journals:
Packer DL, Three-Dimensional Mapping of Intervential Electrophysiology: Techniques and Technology. Journal of Cardiovascular Electrophysiology 2005; Vol 16, No. 10, 1110-1117
Packer DL: Evolution and mapping and anatomic imaging of cardiac arrhythmias. J Cardiovasc Electrophysiol 2004; 15: 839-854
Hocini M, Sanders P, Jais P et al. Techniques for Curative Treatment of Atrial Fibrillation. Journal of Cardiovascular Electrophysiology, Vol. 15, No. 12, December 2004, p 1467.
Oral H, Pappone C, Chugh A. Circumferential Pulmonary Vein Ablation for Chronic Atrial Fibrillation. NEJM 354:9, Mar. 2, 2006, p 934.
Nademmanee K, Mckenzie J, Koar E, et al. A New Approach for Catheter Ablation of Atrial Fibrillation Mapping of the Electrophysiologic Substrate. JACC Vol 43, No. 11, 2004. p 2044.
Gonzalez M D, Otomo K, Shah N. Transeptal Left Heart Catehterization for Cardiac Ablation Procedures. J Interventional Cardiac electrophysiology 2001. 5, 89-95.
Pappone C, Santinelli V. The Who, What, Why and How-to Guide for Circumferential Pulmonary Vein Ablation. J Cardiovascular Electrophysiolgy 2004. Vol 15, 1226-1230.
Padeletti L, Barold S S. Digital Technology for Cardiac Pacing. Am J Cardiolo 2005; 95: 479-482.
Thomas J D, Greenberg N L, Garcia M J. Digital echocardiography 2002: now is the time. J Am Soc Echocardiograpy 2002; 15: 831-8.
Feignebaum H. Digital echocardiography [review]. Am J Cardiology 2000; 86: 2G-3G.
Wittkampf F H M et al. LocaLisa, New Technique for Real Time 2 Dimensional Localization of Regular Intracardiac Electrodes. Circulation 1999; 99: 1312-1317.
Packer D L, Three-Dimensional Mapping of Interventional Electrophysiology: Techniques and Technology. Journal of Cardiovascular Electrophysiology 2005; Vol 16, No. 10, 1110-1117.
Packer D L: Evolution and mapping and anatomic imaging of cardiac arrhythmias. J Cardiovasc Electrophysiol 2004; 15: 839-854.
Hsu, J W R et al. Directed spatial organization of zinc oxide nanorods. Nano Lett. 5, 83-86 (2005).
Yoshida N et al. Validation of Transthoracic Tissue Doppler Assessment of Left Atrial Appendage Function. J Am Soc Echocardiography 2007; 20: 521-526
Gonzales M D et al. Transeptal Left Heart Catheterization for Cardiac Ablation Procedures. J Interventional Cardiac Electrophysiology 5, 89-5, 2001
Solomon J H, Hartmann M J. Robotic whiskers used to sense features. Nature 2006, vol 443, 525
Qin Y, Wang X, Wang Z L. Microfibre-nanowire hybrid structure for energy scavenging. Nature. Vol 451, Feb. 14, 2008. 809-813

### SUMMARY OF THE INVENTION

Open communication between sensors that are permanently or temporarily implanted within the heart and conventional cardiac diagnostic imaging equipment is paramount for the design of future generation implanted cardiac devices and intra-cardiac sensors. Improvements in the production of intra-cardiac sensors will depend in part on data transfer between diagnostic imaging equipment that acquires analogous signals with differing technologies. Open connectivity between extra-cardiac (or extrinsic) equipment acquiring sensor signals (e.g. echocardiographic indices) and intra-cardiac (or intrinsic) devices (e.g. implanted monitors/devices, diagnostic catheter based systems) enables input and updating of data within the software algorithms of intrinsic systems. This updating process is capable of calibrating and programming intrinsic devices/apparatuses.

Comparisons of analogous data sets acquired intrinsically and extrinsically in various patient sub-groups enable accurate calibration of novel sensors within intrinsic systems. This calibration is dependent on cross-correlation of acquired data in order to appropriately scale the derived physiologic indices. An appropriate set of values is assigned to intrinsically acquired sensor signal data with a range between most pathologic and most physiologic (toward normalcy) based on such cross-correlation.

Bi-directional data transfer (open connectivity) between said extrinsic and intrinsic systems also directs programming of intrinsic systems and provides valuable intrinsic diagnostic data to extrinsic systems. In turn, this data is used to provide prognostic information to the clinician and offer treatment options. The power of the system is dependent on the number of patients who have access to it. Its utility will increase as greater volumes of bidirectional wireless data transfer occur between intrinsic and extrinsic devices to and from peripherally and centrally located processing centers. Communication of information to the clinician can be in a variety of formats including via device based programmers, computer terminals or in a preferred mode of the invention at the level of the intrinsic system itself. Examples of the latter include implanted cardiac devices/programmers (e.g. cardiac electrical stimulation devices as described in the parent application), and other lead/catheter based systems. In order to describe the most advanced and preferred mode of the invention, we will describe its application to a specific cardiac sensor design; a microfabricated implanted catheter/lead based motion sensor with tactile feedback.

### BRIEF DESCRIPTION OF THE DRAWINGS

FIG. 1 shows a block diagram for handling information in accordance with this invention.

FIG. 2 illustrates how an analog piezoelectric signal is A/D converted and communicated to peripheral (400) and central (1000) processing centers as well as conventional imaging equipment (700).

FIG. 3 illustrates analogous signals of cardiac motion obtained with intra-cardiac sensors (middle) and tissue Doppler echocardiography (top—longitudinal motion, bottom—rotational motion).

FIGS. 4A and 4B depict nanosprings and multiple ZnO nanosensors deployed in three dimensions in the distal portion of an intra-cardiac catheter, respectively.

FIG. 4C left is an electron micrograph of an individual ZnO nanowire and 4C—right illustrates how nanowires are radially positioned about a Kevlar fiber core mechanically reinforced with layers of TEOS (see text for details).

FIG. 5 illustrates similarities between tissue Doppler imaging (TDI) assessment of left atrial wall motion during atrial flutter (Fl) on the bottom, current, I (top) and voltage, V (middle), generated by a piezoelectric sensor.

FIG. 6 demonstrates the relationship between frequency of piezoelectric sensor deformation and the amount of generated current.

FIG. 7 illustrates how the presence of myocardial ischemia can impact the current generated by a myocardial piezoelectric sensor.

FIG. 8 depicts a linear correlation between current and mm displacement of a piezoelectric sensor.

FIG. 9 shows how integration of tissue velocity (top) derives tissue displacement (bottom) as a function of time during one cardiac cycle.

FIG. 10 illustrates the multi-dimensional motion of a catheter's haptic handle and its relationship to generated current from a piezoelectric sensor deployed in the distal portion of the catheter (x axis not illustrated).

FIG. 10a depicts tissue Doppler motion (top) and piezoelectric sensor current (middle) within the left atrial appendage.

FIG. 10b is a transesophageal image illustrating the proximity of the left upper pulmonary vein (PV) to the LAA.

FIG. 10c illustrate the degree of tissue motion as the region of interest moves from the LAA (maximal) into the PV (minimal)—see 10b for abbreviations.

FIG. 10d compares the motion of an intravascular transducer within the LAA (top) and in the os of a pulmonary vein (bottom) subjected to venous flow.

FIG. 11 illustrates a haptic catheter handle's motion in three dimensions and associated ZnO nanowire constructs which are preferentially affected by tissue motion in specific vectors.

FIG. 12 demonstrates how signals from a piezoelectric nanosensor is processed and transmitted to other equipment.

FIG. 13 illustrates how a haptic handle buffers the force from an operator's hand at a critical point.

FIGS. 14a and b illustrate transeptal puncture of the inter-atrial septum.

FIG. 15 depicts catheter force over time during transeptal puncture with varying applied force by the operator (m—medium, f—fast, s—slow).

FIG. 16 illustrates a three dimensional piezoelectric nanosensor (PzNs) and proprioceptive whiskers (PW) with strain gauges (SG) at the distal portion of a cardiac catheter.

FIG. 17 shows a piezoelectric nanofabric (PzNf) positioned over the left ventricle (dotted arrows) with bidirectional communication via carbon nanotube (CTNC) and conventional conductors (CC) to an implanted cardiac device, 5000. PzNf is a bioelectric interface capable of delivering pacing and defibrillation current to the heart and also generates current for energy harvesting (IpZn) and data collection (I data). CNTC hybrids with conventional lead conductors (HC) for ease in connection with 5000. Data collected is available for wireless communication to other equipment (400h, 700, 1000). See text for details.

FIG. 18 is an electron micrograph of a hybrid circuit (e.g. gold and CNTC)

### DETAILED DESCRIPTION OF THE DRAWINGS

In this invention, temporary or permanently implanted devices (e.g. cardiac catheters or implanted pacemaker/defibrillator leads, respectively) are equipped with transducers that acquire sensor signals intrinsically from within the cardiac tissues. By way of example, a piezoelectric sensor acquires information related to the motion of the contacted cardiac tissues and regional intra-cardiac blood flow. The motion and/or deformation of the sensor are directly proportionate to that of the neighboring tissues. The amount of piezoelectric voltage generated will bear a relationship (i.e. linear, exponential) to sensor motion/deformation. Physiologic indices that can be derived from such intrinsically acquired data include but are not limited degree of displacement, frequency of motion (can be along specific vectors), anatomic localization, sensor orientation. These indices are described in more detail within patent application Ser. No. 11/334,935 and below. These indices are applied to provide a haptic control system for navigating about the heart, performing therapeutic procedures and collecting physiologic information.

These physiologic indices provide clinically relevant information such as risk of stroke (e.g. left atrial appendage tissue motion or blood flow) and cardiac mechanical function (e.g. cardiac performance based on regional ventricular wall displacement). This information is communicated to processing centers at various locations which can be peripherally located (e.g. doctors' offices) or centralized centers managed by health care providers, insurance companies, academic institutions and the like. Data is compiled at these centers and analyzed (e.g. research purposes). Outcome information in select patient sub-groups (i.e. based on demographic and/or genotypic classes) subjected to various treatment regimens is ultimately acquired. This outcome information or prognostic data can then be communicated back to peripheral processing centers as well as patients' implanted devices. The clinician will then have access to volumes of valuable data and incorporate this into their decision-making in regard to an individual patient's management.

By way of example, such informational data sets can be in the form of factual statements; of 1450 patients that fall within the same subgroup (e.g. age, ultrasonic findings, demographics, genotypic features, medication regimen, etc.) receiving treatment regimen A, 55 percent more had a more favorable outcome than those receiving treatment B. Favorable outcome is defined, for example, as a composite end-point such as all cause mortality and hospitalization frequency.

In order to appropriately assign a physiologically relevant value to acquired intrinsic sensor signals we compare and correlate this data to that acquired by conventional techniques extrinsic to the evaluated organ system (e.g. echocardiographic Doppler assessment of cardiac blood flow or tissue motion). These comparisons can be between one of more patients or even based on animal data. Data input can be via alternate means and may even be manually entered into data banks within implanted devices, extrinsic diagnostic/therapeutic equipment, EMRs and processing centers. In the preferred mode of the invention, the data input occurs via wireless telemetry. Ideally, patient outcome data is determined with a consideration of physiologic indices based on intrinsic sensor signals acquired. Once volumes of data sets are accrued from patient sub-groups, prognostic information can be derived and communicated back to the clinician.

Implanted Catheter/Lead Based Motion Sensors for Diagnosis and Treatment of Cardiac Arrhythmias

In implanted cardiac rhythm management (CRM) devices and intra-cardiac catheters, first generation lead based piezoelectric sensors and three-dimensional navigational systems are capable of characterizing tissue motion and determining anatomic properties, respectively. In order to understand more thoroughly the workings of this invention, the inventor applies the sum and substance of his concepts to the design of novel intra-cardiac nanosensors capable of detecting anatomic properties and motion of cardiac tissues. Such sensor nanotechnology will optimize data acquisition and communication of meaningful physiologic data above and beyond the systems in use today.

Though the current systems (see Description of Prior Art) help guide localization of intra-cardiac catheters and are a dramatic improvement over conventional fluoroscopy, they do not obtain clinically useful diagnostic information about cardiac mechanical function or catheter-tissue contact, nor are they standardized and calibrated relative to other patients. Advances in open connectivity coupled with nanosensor technology are needed to accomplish these tasks.

In one mode of the invention, the anatomic data collected by these systems are used not only to generate clinically relevant data such as characteristics of regional wall motion (e.g. atrial appendage function) but also to more accurately localize the catheter tip in relationship to cardiac tissues receiving therapy (e.g. ablation). Combining and comparing a plurality of intrinsically and extrinsically acquired data sets from an indwelling catheter (intrinsic) and that obtained with other extrinsic diagnostic modalities (e.g. echocardiography, CT angiography, magnetic resonance imaging, LocaLisa, NavX) enables the system to assign values to any of the intrinsically derived motion indices based on correlations with analogous sensor data using conventional techniques. Thus a plurality of signals can be acquired and compared to accomplish the task of data collection. The more data collected and analyzed the more powerful the system will be at providing clinically useful information to the clinician, offering treatment options and directing therapy. The information can be communicated to the health care provider as raw data or as an image (e.g. parametric imaging).

In a preferred mode of the invention, motion and anatomic information is communicated using a piezoelectric-based tactile feedback system present in the handle of a cardiac catheter (e.g. ablation catheter) or intra-cardiac lead at time of implant. The data collected by the system yields information including but not limited to intra-cardiac chamber anatomy, tissue vibration frequency/amplitude/vector.

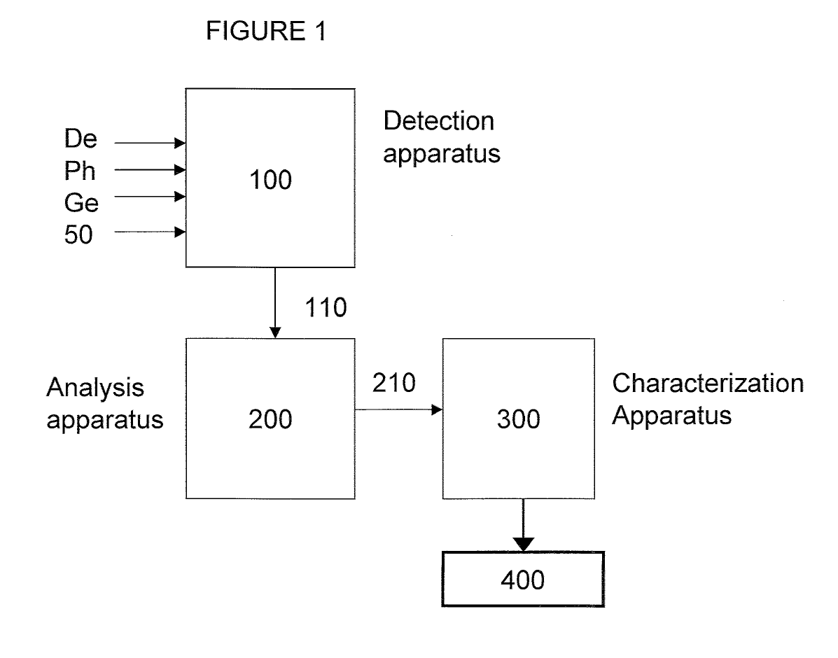

FIG. 1 shows a block diagram for handling information in accordance with this invention and demonstrates how collected intrinsic and extrinsic data is used to provide prognostic information, direct treatment and communicate data to the clinician as it applies to treatment of the most common cardiac arrhythmia, atrial fibrillation. Chronic, background data such as an individual patient's demographic (De), phenotypic (Ph) and genotypic (Ge) information is entered into 100. These data are compared to pooled data acquired from other patients which is also input into the system (50). These data are input at step 110 into 200, analysis apparatus. At 200 the data is used to categorize an individual patient within subgroups of patients who share similar characteristics (e.g. age, sex, race, family history, personal cardiac history, genotypical features). Some of these characteristics are scored a numerical value based on degree of pathology, and some of these characteristics can be either on or off (e.g. male or female, absence or presence of coronary artery disease). By way of example, one subgroup is defined as having a low likelihood of developing a stroke from atrial arrhythmia based on the absence of certain familial characteristics and hematologic indices not associated with thrombus formation. The details of how this is accomplished can be found in the parent patent application and references provided.

Discriminant analysis or other techniques can be applied to incorporate more than one clinical parameter into the final set of indices that characterize an individual patient. In order to provide such detailed clinical data to the clinician, the data is input into a characterization apparatus at step 210 and analyzed at 300 along with a combination of data which may be acquired intrinsically and extrinsically. The data input into 300 include the chronic datasets as well as more acute information that is subject to change (as opposed to the chronic data input into 100). This acute data can be acquired in real time with continuous updates. By way of example, this data can include sub-acute information related to cardiac structure (left ventricular mass) and function or acute data such as the degree of left ventricular torsion while inotropic electrical stimulation (e.g. cardiac contractility modulation) is applied to myocardium as described in the parent application. This data can be collected in real time and even updated with each cardiac cycle. A numerical value is assigned for all characteristics in binary format. All data relates to one or more relevant physiologic properties. Communication to the clinician occurs at 400. Such communication can occur in a variety of ways as described below.

Cross-correlation of analogous intrinsic and extrinsic data sets enables accurate interpretation of intrinsically acquired data and facilitates the design and calibration of novel intrinsic sensors. Ultimately, much of the data is obtained intrinsically, by the catheter or implanted lead and cardiac system itself, without a need to obtain data from outside sources (e.g. echocardiography). In order to assign a clinically relevant numerical value descriptive of degree of pathology for data obtained with novel intrinsic cardiac sensors, comparisons may need to be made from time to time with analogous data sets obtained extrinsically.

### Piezoelectric Sensors

In one mode of the invention, intra-cardiac motion sensors detect properties of tissue displacement. Such displacement can include the natural motion/deformation of cardiac tissues and/or displacement caused by a catheter or lead based system in contact with the heart and blood vessels.

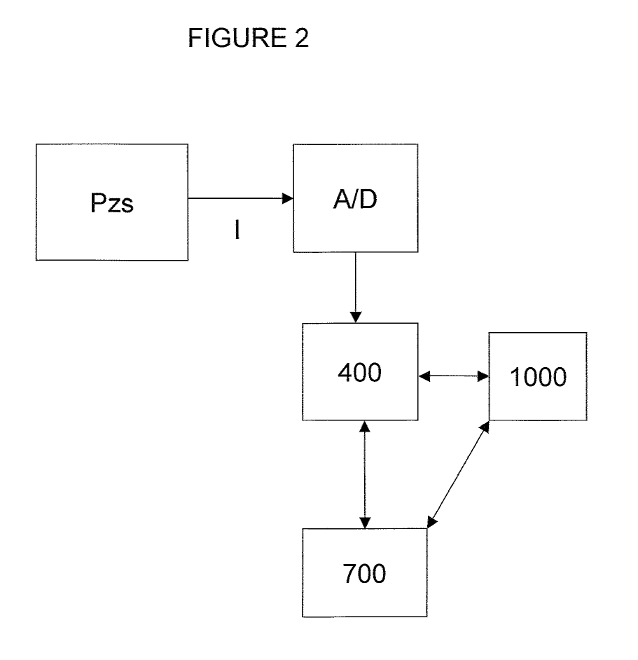

In a simple format, the frequency of motion of the tissue (e.g. left atrial appendage) is detected by lead based transducers such as a piezoelectric sensor (Pzs in FIG. 2). The analog data acquired by the sensor is in form of voltage, V, or current (I in FIG. 2) generated by deformation of Pzs. Frequency, vector and degree of displacement are examples of information that can be quantified. Any piezoelectric material may be implemented to accomplish this task and is known by those experienced in the art (e.g. accelerometers) and described in the provided references. The analog data is digitized. This can be accomplished at the level of the lead/catheter system itself or in a separate apparatus (A/D in FIG. 2). This is then communicated to the operator at 400 in one or more formats. 400 can be a video display similar to that seen with conventional echocardiographic imaging technology (e.g. tissue or pulse wave Doppler). In a preferred mode of the invention, 400, is a tactile feedback system contained within the handle of the indwelling catheter or lead. The utility of such a tactile feedback system is best understood by those familiar with cardiac catheterization procedures.

The information communicated serves multiple purposes. Foremost, it provides important diagnostic information about the characteristic motion of cardiac tissues. For example, LAA motion is related to stroke risk. Left atrial wall contraction velocity has been found to identify patients at risk for cerebral embolism. (Yoshida N et al. Validation of Transthoracic Tissue Doppler Assessment of Left Atrial Appendage Function. J Am Soc Echocardiography 2007; 20: 521-526). Myocardial motion is also known to relate to infarct extent, systolic and diastolic cardiac performance.

In a preferred mode of the invention, such a piezoelectric system is “taught” the relationship between voltage or current amplitude, frequency and vector by having analogous measurements made using conventional technologies (e.g. tissue Doppler via transesophageal or intracardiac echocardiography) at 700 in FIG. 2, correlated to those derived from intra-cardiac sensors at 400. This “teaching” process (e.g. neural networks) may occur at 400, 700 or a central processing center at 1000. Analogous measurement data can also be supplied by an intra-cardiac navigational system as the extrinsic educational tool at 700. Thus, a plurality of signals can be input and compared as to calibrate an intra-cardiac sensor (e.g. piezoelectric transducer).

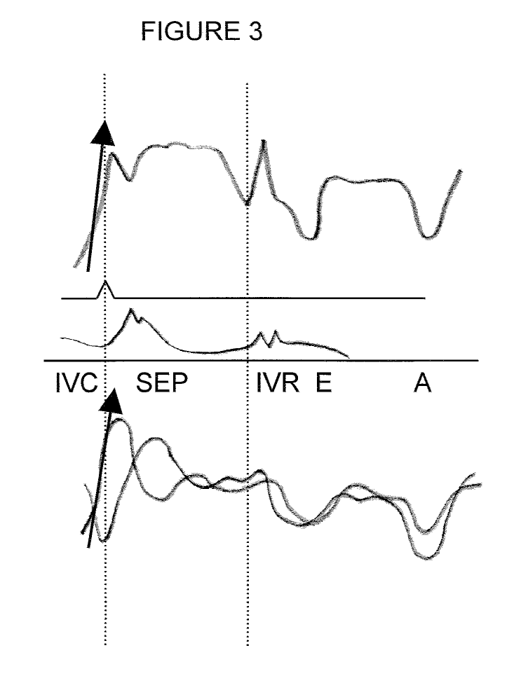

Referring to FIG. 3—middle, we see a current time graph illustrative of lead or catheter motion at the level of the atria-ventricular valvular annulus (along the plane of the coronary sinus) detected by an LV lead accelerometer. The lead has Pzs incorporated within its structure. Optimally, the lead/catheter remain isodiametric and in a preferred mode of the invention, Pzs is constructed with nanotechnology (e.g. carbon nanotube transducers). On the bottom of FIG. 3 is a rotational displacement time graph depicting left ventricular torsion as determined by echocardiographic speckle tracking or other imaging technique. On top is ultrasonic tissue velocity. One heart beat is depicted. Current peaks are noted at times of maximal rotational velocity and displacement during isovolumic contraction (IVC) and isovolumic relaxation (IVR). Less current flow is noted during the systolic ejection phase and diastolic time frames (E=passive filling and A=active filling).

Pzs in this example is constructed using nanotechnology, PzN. Such a sensor design is expected to not only provide a high fidelity signal, but also generates a relatively large amount of current relative to degree of deformation. This improves the signal to noise ratio. In one mode of the invention, the current generated is used not only for data collection, but also is stored and helps supply power to the system (see section on Nanogenerator below). Such an application is optimal for increasing battery longevity for the implanted device.

### Piezoelectric Nanosensor

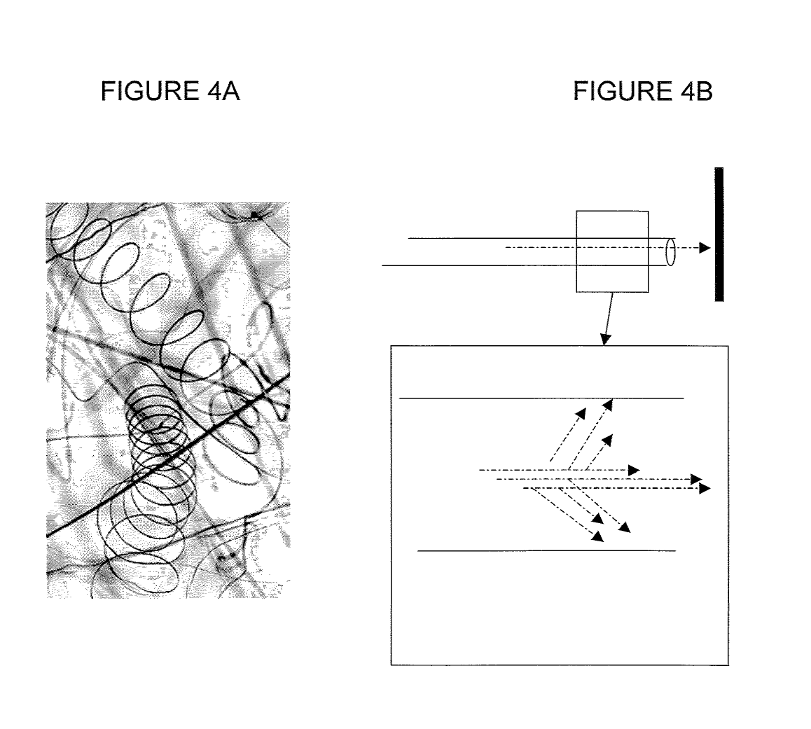

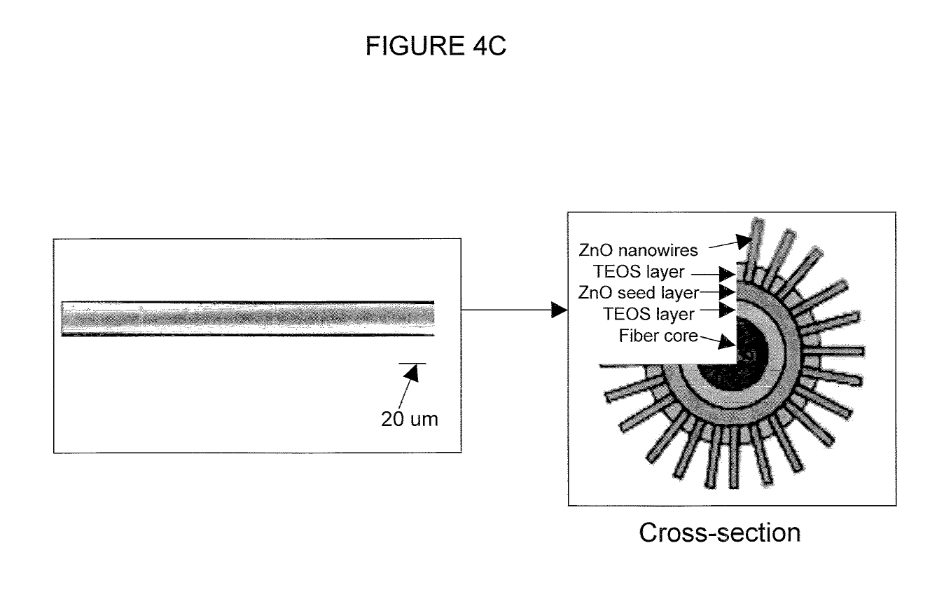

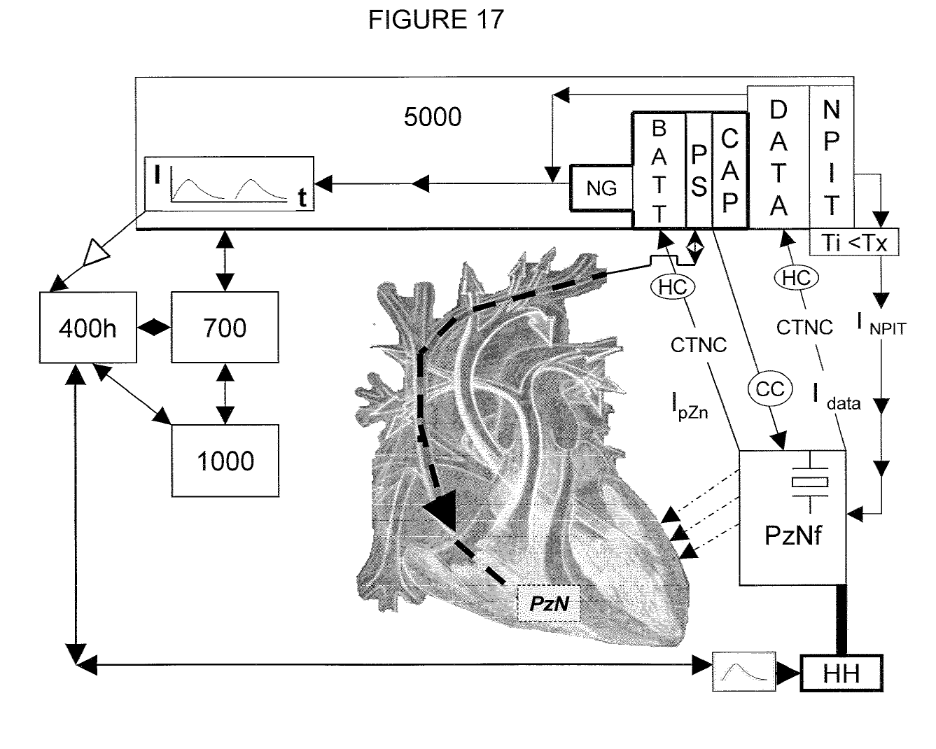

Nanometer-scale structures that spontaneously form helical shapes from long ribbon-like single crystals of zinc oxide (ZnO) or “nanosprings,” (presented at the 227th national meeting of the American Chemical Society in Anaheim, Calif.) have piezoelectric and electrostatic polarization properties that make them useful in small-scale sensing and micro-system applications (FIG. 4a). Carbon nanotube fiber constructs have been found to be biocompatible with mammalian cells and neurons and serve as electrical conductors that interface with neural tissue and the like (Dubin et al. Carbon nanotube Fibers are Compatible With mammalian Cells and Neurons. IEEE Transactions on nanobioscience, Vol. 7, No. 1, March 2008). In one preferred PzN design, gold-coated zinc oxide, ZnO, nanowires are grown radially on Kevlar 129 fibres using a hydrothermal approach (Hsu, J W R et al. Directed spatial organization of zinc oxide nanorods. Nano Lett. 5, 83-86 (2005)). This structure is a microfibre that is embedded within the distal portion of a catheter or lead (e.g. circumferentially about or within the insulation). The ZnO nanowires (dotted arrows depicted in FIG. 4b) are single crystalline with a hexagonal cross-section and diameter ranging between 50-200 nm and a length of 3-4 um. Spatial distance between the nanowires is of the order of a few hundred nanometers to allow them to bend and generate piezoelectric potential. A surface coating of tetraethoxysilane (TEOS) maintains flexibility and improves the long term mechanical performance of the PzN after growth of the crystalline film and nanowires. The interspace distance between the nanowires is directly proportionate to the length and diameter. The tips of the nanowires are separated from each other by a tilting angle of <+/−10 degrees and the bottom ends are tightly connected (FIGS. 4b and c). The bottom ends are a common electrode for signal output and are in form of a continuous conductive film. This end is connected to a conductor (FIG. 17) which can be a conventional conductor (CC) as commonly found in implanted leads/electrodes or a carbon nanotube conductor (CNTC). Details of the construction of such a microfiber-nanowire hybrid structure can be found in the scientific literature (Qin Y, Wang X, Wang Z L. Microfibre-nanowire hybrid structure for energy scavenging. Nature. Vol 451, Feb. 14, 2008. 809-813). In this application of the invention, multiple PzN are deployed at the distal portion of an implanted lead/catheter (FIG. 4b) or can even be woven into the fabric of carbon nanotube ribbons, nanofelts or scaffolds that are implanted along the cardiac surface (e.g. eipcardial/pericardial). In one mode of the invention, the carbon nanotube fiber constructs can act as active electrodes for pacing and defibrillation. The precise means for constructing such a PzN herein is exemplary and other microfabrication techniques are within the scope and spirit of this invention.

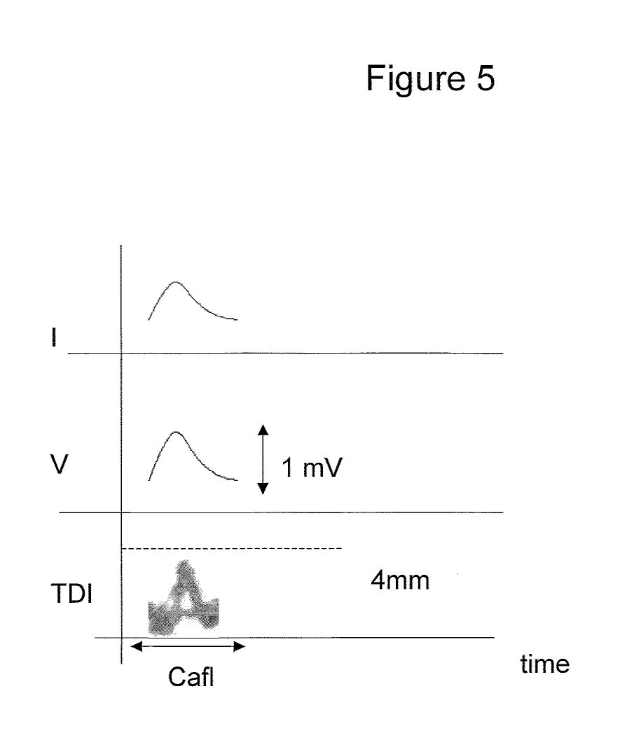

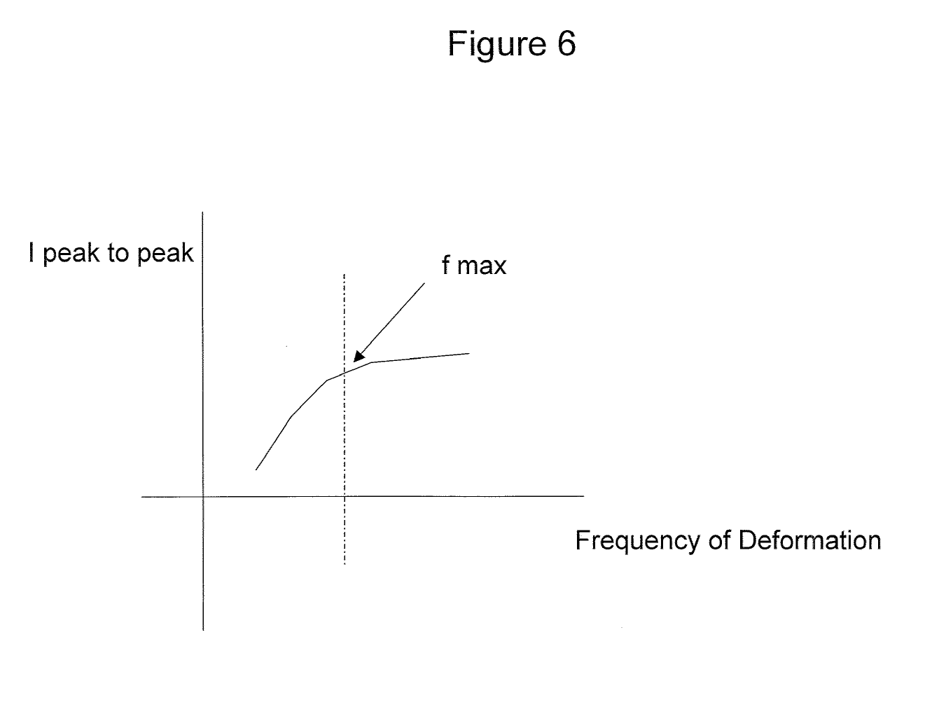

Electrical output of PzN is depicted in FIG. 5 as current (top) and voltage (middle). Cyclical generated energy is evident in this example, where one period is associated with each cycle in a patient with atrial flutter detected with echocardiographic tissue Doppler imaging, TDI. The peak displacement of the catheter tip within the atrial appendage (e.g. RAA in a pacing lead or LAA in an ablation catheter) can also be derived using other extrinsic methodologies (e.g. fluoroscopic analysis, 3D navigational assessment with externally applied electric fields). To better understand the workings of the invention, tissue Doppler assessment of one cycle of left atrial appendage motion during atrial flutter is depicted in FIG. 5 on the bottom (Cafl) along with PzN current (top) and voltage (middle). Peak displacement of 4 mm is noted with TDI. Peak to peak voltage generated is approximately 1 mV. Thus PzN determined displacement, PzN mm=4*PzN mV. By way of example, measurements obtained in this and other patients demonstrate a linear relationship with a slope of 4 mm/1 mV until the PzN is unable to generate incremental voltage secondary to mechanical characteristics of PzN which preclude near complete recovery of cyclical deformation and increases in current output (FIG. 6). At a specific frequency for any given construct the system will not be useful for monitoring data. This can be determined by correlations with an extrinsic modality.

By way of example, digital fluoroscopic imaging of catheter motion will allow acquisition of two-dimensional deformation frequency. This can be compared to I (t) dt and when the association is no longer relatively linear the system will not be clinically applicable. One can infer that the frequency of motion is above some value f.sub.max when the system approaches this asymptotic point (FIG. 6). Preferably, the construction of PzN is such that optimal operation occurs within specific bandwidths. Thus, a catheter used for ablation of atrial arrhythmias will operate with frequencies between 50 and 500 cycles/minute and one used for ventricular therapies (e.g. pacing lead or ablation catheter) may operate optimally between 5 and 250 cycles/minute. Modification in the specifications of the nanowires and changes in the interspacing between nanowires is used to achieve optimal material characteristics.

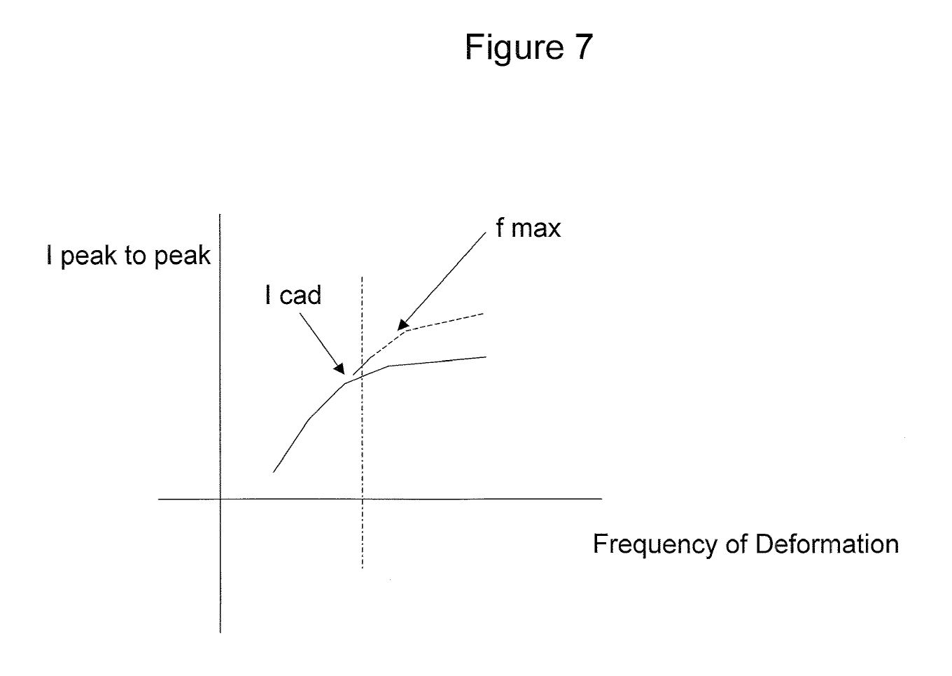

The importance of understanding the frequency fatigue point of various PzS and PzN can be understood by looking at FIG. 7. In this patient or patients (averaged data) the current generated is limited by coronary insufficiency and I becomes asymptotic at a point before f max would be reached. Thus, the current generated is limited by limited deformation secondary to myocardial ischemia and not transducer properties (FIG. 6). For this particular intrinsic sensor it is known that f max occurs at a displacement of approximately 18 mm. Peak normal ventricular myocardial (longitudinal) displacement is 12 mm along the base of the left ventricle. Greater values can be found in hyperkinetic myocardium, but generally will not exceed 15 mm. By way of this example, the importance of cross-correlation of intrinsic and extrinsic data, system standardization and calibration in the design of novel sensors is clear and enables accurate nanosensor detection of myocardial ischemia.

The motion data acquired can be stored in 400 which in various applications of the invention are contained within the implanted device, device programmer or as part of a tactile feedback system (described in more detail below). The latter application will help the operator position an intra-cardiac catheter or lead system and provides diagnostic information in a novel format. The collected data is communicated to a central processing center along with the data collected from other patients. In an advanced application of the invention, a plurality of intrinsically and extrinsically acquired data (chronic and acute) is implemented as part of closed feedback systems within the cardiac device which operate to activate specific pacing schemes in select patients at the appropriate time frames (e.g. initiate inotropic electrical stimulation in select patient subgroups at appropriate times which is discussed in the parent application).

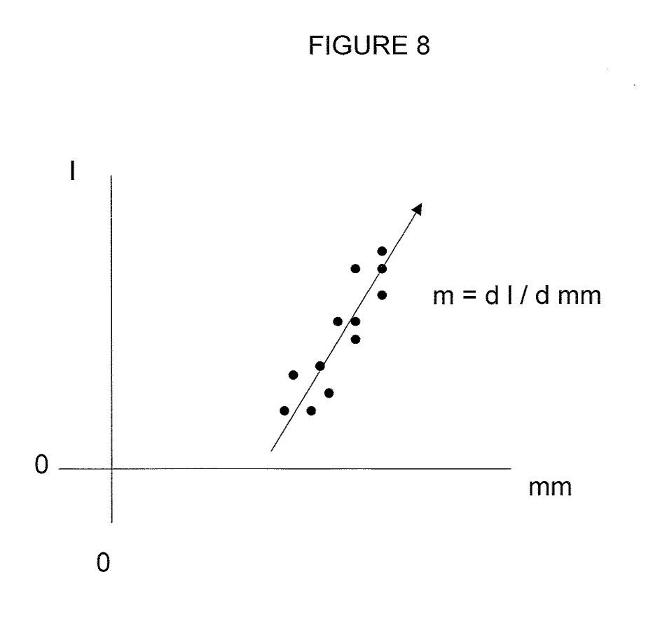

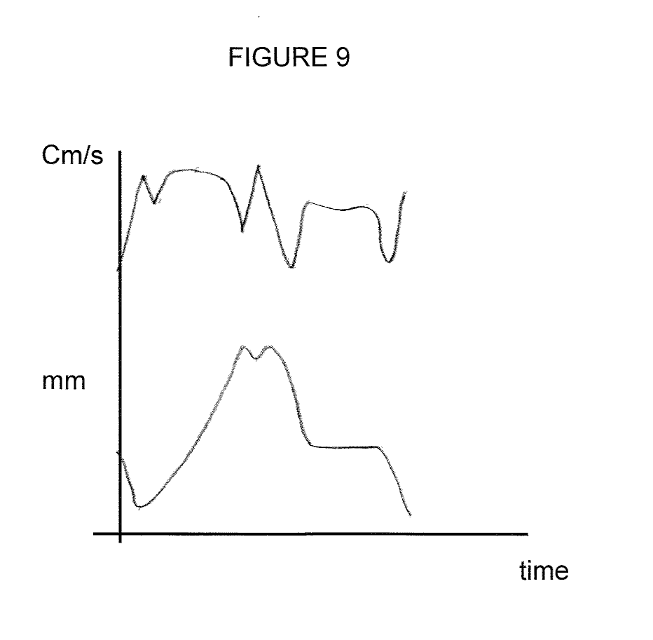

Referring to FIG. 8 we see a scatter plot demonstrating peak catheter tip displacement, mm, (along the left ventricular endocardium) determined using an extrinsic navigational system such as that generated by injection of an externally applied electrical field for real-time three dimensional localization based on voltage drops. As mentioned above, localization accuracy of such systems is less than 2 mm. Alternatively, tissue Doppler assessment or other echocardiographic parameter of peak systolic displacement can be implemented (FIG. 9). Referring to FIG. 8, on the abscissa is peak systolic displacement (e.g. average over a number of cardiac cycles). Myocardial wall motion displacement values during cardiac systole approach 12 mm in normal subjects. On the ordinate is peak current generated by Pzs for the same patient. Each point represents peak displacement and peak current. The graph can be acquired by obtaining multiple measurements for an individual or even for multiple individuals. This data can be collected manually, semi-automatically or in a preferred mode, via open connectivity for all patients that have access to this technology. Initially, laboratory derived data based on animal studies can be used to understand the relationship between extrinsic and intrinsic modalities and for initial sensor design. In this example, the relationship between Pzs current generated and displacement determined extrinsically via a navigational system is linear. Patients with normal myocardial wall motion (data points toward x, y=maximal values) and those with advanced cardiomyopathy (data points toward x, y=0,0) are included.

A similar scatter plot for displacement of catheter tip within the LAA can be derived. Patients with normal sinus rhythm (toward x, y=maximal values) and those with atrial fibrillation (toward x, y=0) are included. Such wall motion can be radial, longitudinal, rotational/torsional (0-30 degrees) or a combination of vectors. For atrial appendage motion, normal frequency range is 40 to 90/minute and abnormal is 100-500/minute. Atrial appendage displacement can vary between 1 and 3 mm in normal subjects and less than 1 mm under pathologic circumstances (e.g. during atrial arrhythmia). Relationships between displacement and frequency can be obtained for analysis as well.

In this example (FIG. 8), the relationship between intrinsically (PzS) and extrinsically acquired signals (e.g. NavX, tissue Doppler) is linear and is solved by the equation y=m×+b, where y=peak systolic current generated by Pzs=I and x=peak systolic displacement=mm. As illustrated b=1.5. The relationship between I and mm remains linear +/−10 percent. If a correlation with error >15% (derived from the scatter plot) exists between intrinsic and extrinsic indices, the system indicates that a large margin of error exists and that correlations between the two methods are not accurate. Under such circumstances, the intrinsic sensor data can still be valuable but can not be equated to more commonly used metrics.

Under ideal circumstances (e.g. margin of error <5%), a common mathematical index may be used to describe the sensor signals such as percentage of normal (0-100%) based on the range of values acquired (in the parent application this is derived by the translation function). The greater the number of data points the more accurate is the relationship delineated. In this example, and for purposes of simplicity, such a technique assumes linearity of both the measurement techniques and physiologic properties relative to degree of pathology, though correlation between the intrinsic and extrinsic data collected can be solved with an exponential or other non-linear equation.

Preferably, a plurality of signals are used to derive a relationship between intrinsic and extrinsic sensor data. By way of example, anatomic localization data can be based on more than one modality (e.g. echo, MRI, CTA and navigational systems) as well as with piezoelectric transducers, the latter of which may provide optimal wall motion frequency data. Thus, the equation derived may include more than one dimensional variable (e.g. x=Nav data, y=Pzs data, z=tissue Doppler data).

### Communication to the Clinician

The data collected can be displayed as values or an index of pathology (e.g. 40-50% of normal values) and/or as a visual display. The visual display can be reconstructed waveforms that in one mode of the invention are post-processed (e.g. Gaussian filters, noise reduction algorithms) and portrayed as an image easily interpreted by the clinician familiar with echocardiographic Doppler data (e.g. tissue Doppler parametric imaging techniques) comparable to that found on common echocardiographic equipment manufactured by companies such General Electric, Philips, TomTec and the like.

In the preferred mode of the invention, motion/deformation sensor data is communicated to the clinician via a tactile feedback loop within a handle (400h) held by the operator (Haptic Handle). The Haptic Handle, HH, can be contained within conventional handles (e.g. U.S. Pat. No. 6,780,183) used for positioning pacemaker leads, catheters, or intravascular delivery/extraction systems or be a separate system that has bidirectional wireless communication (open connectivity) with intracardiac sensors and processing centers as described above. Simplified tactile virtual reality interface devices are known by those experienced in the art and have been used in game controllers and to simulate medical procedures, for example, for teaching purposes (U.S. Pat. Nos. 5,389,865, 5,769,640).

### Piezoelectric Based Tactile Feedback System

The benefit of the tactile feedback is ease of procedure in addition to data communication and collection as described in detail below. Positioning a right (or left) atrial lead within the RAA (or about the LAA) and being able to feel the motion and confirm appropriate tissue contact will reduce likelihood of dislodgement secondary to suboptimal positioning and also help the operator recognize if cardiac perforation is imminent. The lead or catheter can incorporate micro-fabricated Pzs tranducers using nanotechnology (see below) and have the capacity to generate data to an implanted device. Preferably, communication is sent wirelessly to device programmers/processing centers and communication interfaces providing important physiologic information (e.g. about atrial mechanical function).

When applied to positioning a left ventricular lead or intravascular delivery/extraction system, the diagnostic data will reveal properties of vascular tissue, cardiac wall motion and myocardial mechanics.

The tactile feedback system will assist in positioning intravascular catheters such as a lead extraction system (e.g. laser assisted lead removal) or performing catheter ablation for various arrhythmias. By way of example, the precise location of the tip of the ablation catheter can be determined by sensors that detect cardiac tissue mechanics as a function of time. In a preferred embodiment, a microfabricated Pzs sensor using nanotechnology (PzN) will be able to detect not only left atrial appendage motion but also the texture of the contacted surface, providing anatomic confirmation and never seen before data representative of cardiac structure at an extraordinary resolution. Calculation of the elasticity and “rupture point” of the IAS is an example of how such an invention will have clinical applicability and reduce the risk of procedural complications. In an alternate mode of the invention, implementation of proprioceptive whiskers improves the systems ability to provide anatomic detail (see below).

Displacement information (e.g. degree/vector of displacement, frequency of motion) gathered by the system is uploaded into memory and can be wirelessly communicated to an EMR, device programmer, peripheral or central processing center as specific clinical indices. Cross-correlation of the data with similar data from patients with access to this technology occurs, preferably at the processing centers. Outcome data and other informational data sets are communicated back to the equipments' memory and is provided to an operator or other interested party. The operator will learn to differentiate one individual patient's cardiac tissue mechanical properties from others by using a sense of touch much as clinicians today use auditory cues with a stethoscope. Open connectivity will facilitate this learning curve by allowing the clinician to draw comparisons between patients at multiple centers. Samples of other patient subgroups' tactile characteristics can be remotely uploaded for educational and comparative purposes. Experts at outlying centers can assist in real-time procedures via open connectivity between intra-cardiac sensors, processing centers and communication interfaces. Data communication in form of mathematical clinical indices (e.g. amplitude, frequency, vector of motion), visual displays and tactile feedback will facilitate this process.

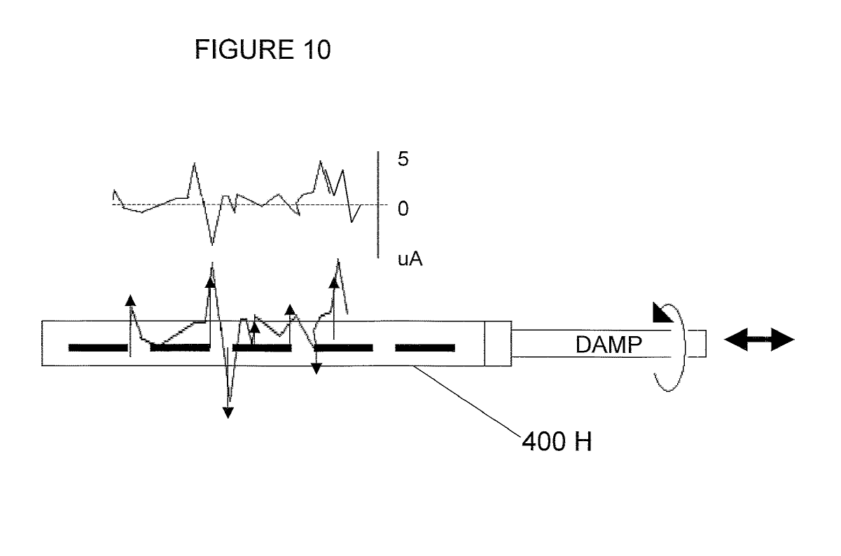

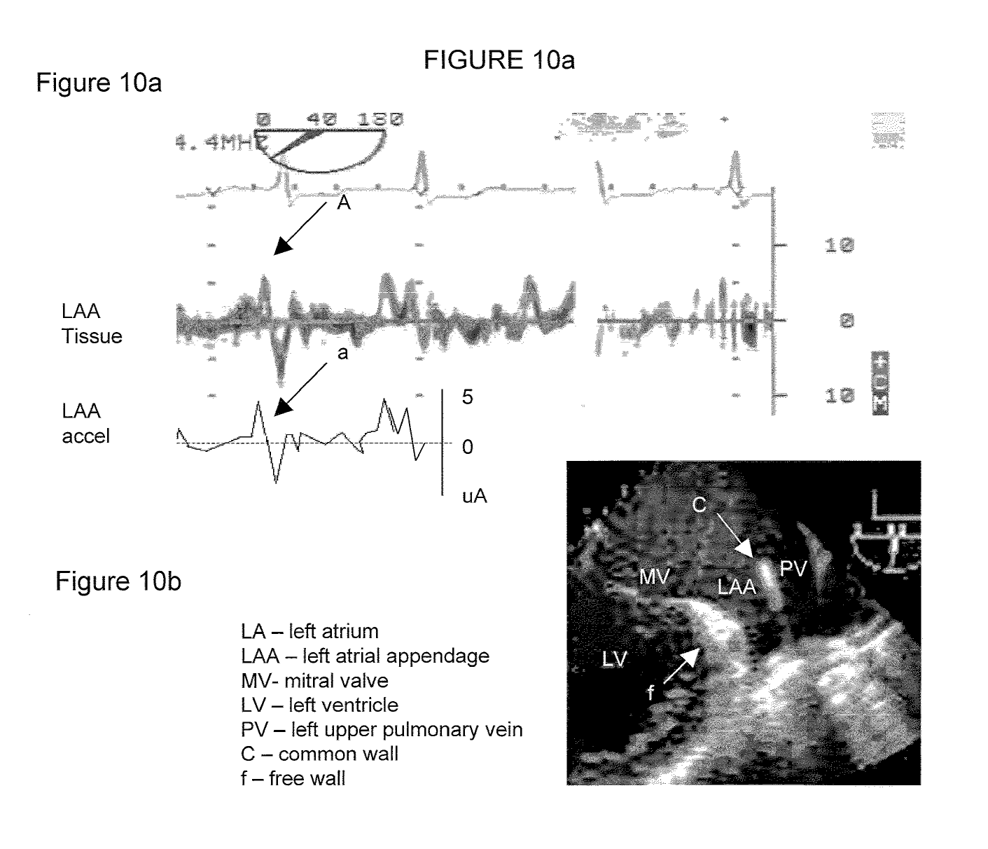

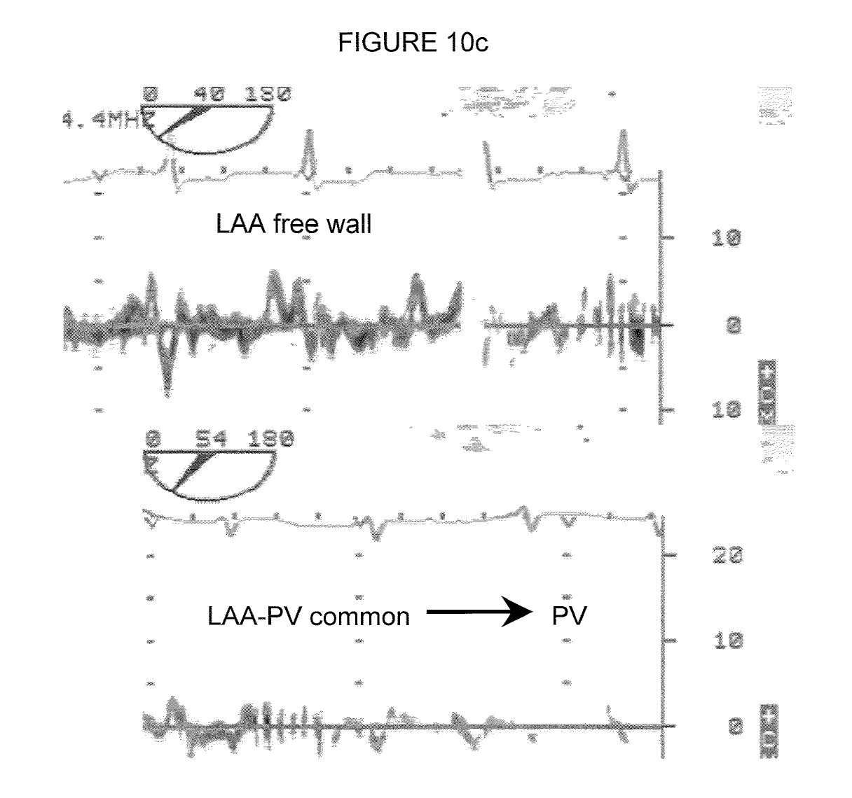

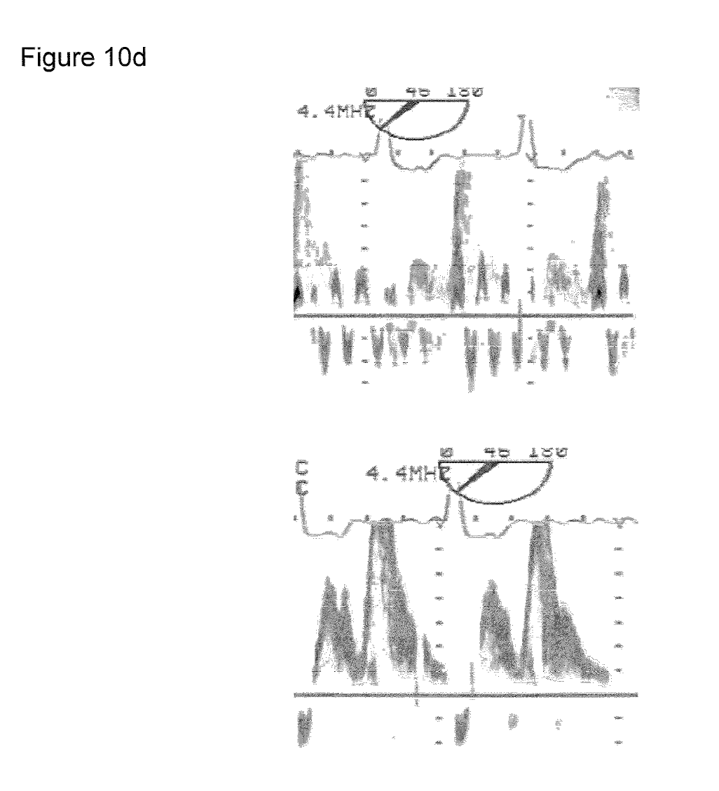

In one mode of this invention, the handle accommodates a tactile feedback motor with shaft and offset weight mounted as a component within the catheter handle for providing vibration/displacement to the hand of the operator, though, other tactile feedback and force feedback handle designs are within the scope and spirit of the invention. The vibration/displacement simulates the motion detected by the intravascular or intra-cardiac PzS or PzN. Thus, the fine, high frequency motion of the LAA is translated into a similar quality motion in the catheter handle, 400H, (FIG. 10). Similarly, the resistive force upon an excimer laser or alternate extraction system is appreciated by the operator.

In FIG. 10, top, we see the current waveform as a function of time derived from PzS and motion perpendicular to the longitudinal axis of a catheter handle (bottom). This motion can also be multidimensional, rotational and to and fro (coaxial). The motion (up and down arrows, y axis) is appreciated along the full length of the handle and is proportionate to the current amplitude (x axis not depicted for simplicity purposes). The gain of the handle's motion is adjustable but always proportionate to catheter displacement at its distal sensor. For optimal reproduction of tissue mechanics, omni-directional vibration/displacement and torque of the catheter tip detected by one or more catheter based PzNs (or other sensors) is transmitted to the handle of the catheter.

By way of example, simulation of intra-cardiac motion is via force feedback using one or more motors housed in the handle. The shaft of each motor holds an unbalanced weight. When power is supplied to the motor, it spins the weight. Because the weight is unbalanced, the motor tries to wobble. But since the motor is securely mounted inside the controller, the wobble translates into a shuddering vibration of the controller itself. The frequency of the vibration and/or degree of handle displacement (i.e. force feedback) is dependent on the current supplied and this current is proportionate to the current generated by PzN or PzS in the distal portion of the catheter/lead.

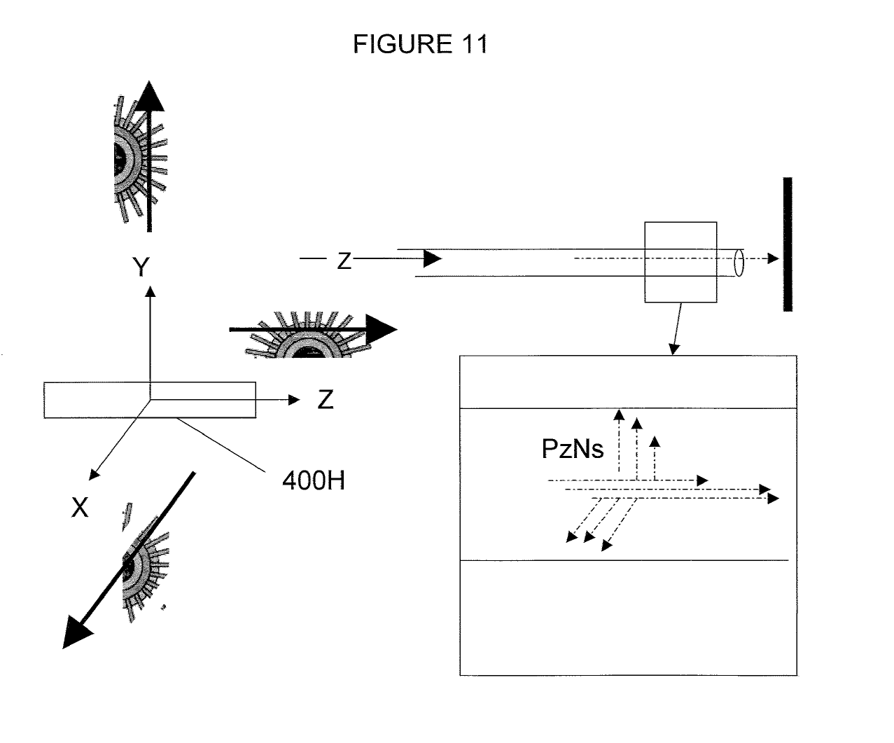

In a simplified mode, amplifiers can appropriately step up the actual current generated by one or more PzN or PzS to the motors within the catheter's haptic handle (see amp and 400h FIG. 17). One of the three sets of PzN are oriented along the longitudinal axis of the lead or catheter (z axis) and two other sets are oriented orthogonal (x, y axes) as illustrated in FIG. 11 by the dotted arrows. The degree of displacement and frequency of displacement of PzN is proportionate to the action provided by the three motors within the catheter handle. In a preferred embodiment, each of the PzN nanowire sets (e.g. ZnO nanosensors) are constructed as to promote fiber motion along specific vectors in three dimensional space (e.g. along longitudinal axis of the catheter tip, Z axis) in order to accurately identify the direction of tissue motion relative to the distal portion of the catheter itself. By way of example, the TEOS layers can be modified as to fixate nanowires along specific axes (e.g. x and y) encouraging motion in one direction (z axis). Alternatively or additionally, CNTC conductors are only ‘innervated’ by the common output of nanowires along a specific sector (e.g. 90 to 180 degrees). Other methods for accomplishing the same are within the scope and spirit of the invention. In FIG. 11 we see three semi-circle ZnO nanowire constructs, each positioned along one axis. Three-dimensional recreation of the motion of the catheter tip is then possible. Phase cancellation of motion characteristics of each set of PzN will be reproduced within the handle and thus the appropriate vector of motion reproduced for the operator. In a preferred mode of the invention, lead based PzN's (e.g. multiple gold coated Zinc oxide PzN's on Kevlar mesh in series for each of three vectors) capable of generating a power supply are used (nanogenerators) and this current is harvested for energy storage in an implanted device. The functioning of such a nanogenerator design is discussed below.

With each set of PzNs arranged in three dimensional space within the lead/catheter corresponding to three controllers (e.g. motors) within the Haptic Handle, tactile feedback can be comprised of multiple sensations similar to that found in commonly used video game controllers manufactured by companies such as Wii, Xbox, Playstation (U.S. Pat. Nos. 6,203,432, 6,641,480, 7,139,621). Periodic vibrations (e.g. LAA fibrillating), texture effects (chordae tendinae, LAA/RAA ruggae), sensations of enclosure (e.g. intracavitary, within pulmonary vein), saturation, stiffness (e.g. free wall), thickness (e.g. interatrial septum), spring effect, deadband, inertia, damper effects, constant force, ramp force and friction (e.g. intravascular), simulation of blood flow (laminar and turbulent) are examples. More sophisticated force feedback systems can be implemented as known by those experienced in the art, and those cited herein are exemplary. Such a tactile feedback mechanism or haptic control system will enable the operator to detect larger scale motions and subtle sensations such as the high frequency signal generated from accelerated bubble formation. Detection of these phenomena will reduce risk of tissue necrosis and perforation.

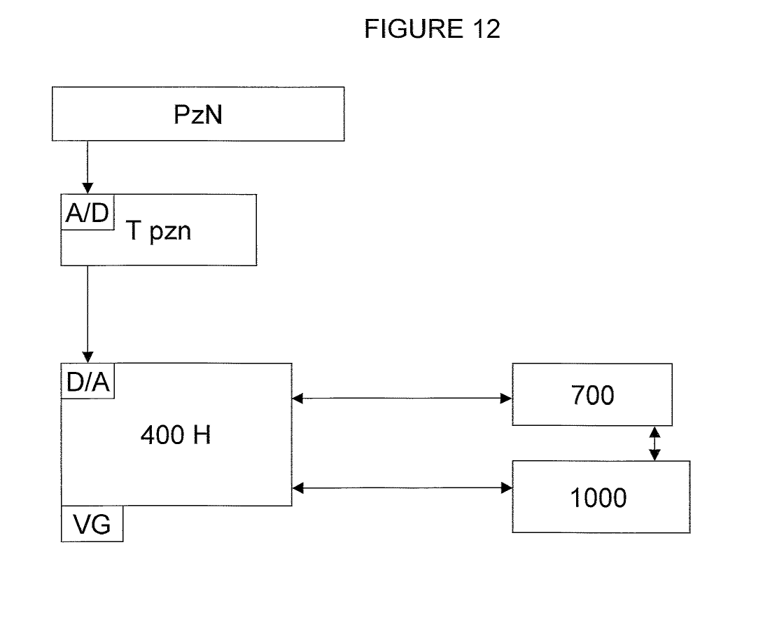

In one mode of the invention, the design of the intra-cardiac tactile feedback mechanism is performed using A/D conversion of the catheter derived PzS or PzN signal and then D/A conversion within the catheter handle (or other connected apparatus) to generate the tactile feedback system. The hardware and software for the tactile feedback system may be in the handle or in a preferred embodiment, in a separate apparatus (T pzn), which is connected to the haptic handle, 400H (FIG. 12). The latter is the preferred mode of the invention as it will reduce the bulk and weight of the catheter handle. The intensity of the tactile feedback motor (vibratory gain (VG)) is adjustable as some operators may desire a more subtle sensation than other operators. Telemetry capabilities are present within such a handle and/or separate apparatus (e.g. TPZN) and allows for wireless transfer/communication of wall motion mechanical data with other equipment (700) and processing centers (1000). Data storage/processing and calibration of system settings is ideally accomplished with bidirectional wireless telemetry (double arrows).

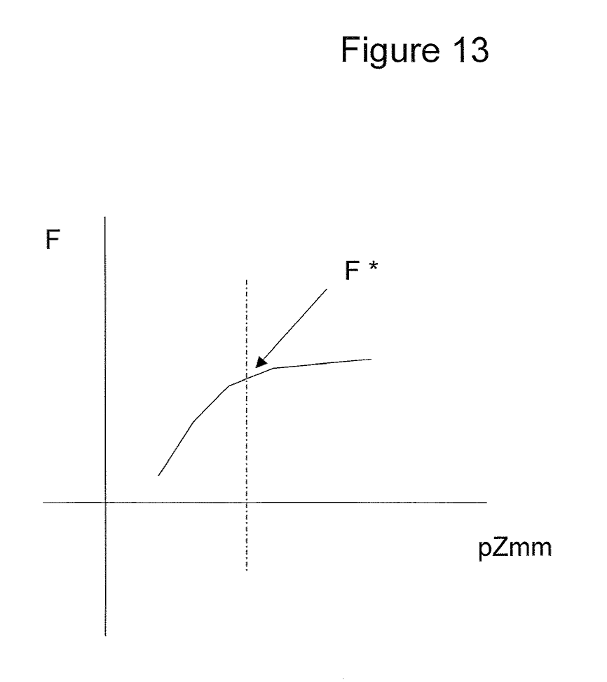

In one mode of the invention, the catheter is connected to the haptic handle and incorporates a dampening or release system. The dampening system allows a buffer between operator manipulation of the catheter and actual tissue contact and the release system disengages handle motion from catheter motion. In addition to the elastic properties of the catheter itself, a dampening system, Damp in FIG. 10, (e.g. spring loaded mechanism) opposed to or within the catheter handle is constructed as part of the feedback motor and shaft/offset weight and simulates the elastic properties of contacted tissues (e.g. vasculature, inter-atrial septum). When the degree of deformation of pZmm (p in FIG. 13) relative to the force generated at the catheter handle becomes non-linear and asymptotic (F* in FIG. 13), the dampening system will provide a buffer (or break the force) as to reduce the likelihood of cardiac or vascular perforation. Any method for designing such a dampening system is within the scope and spirit of the invention and those mentioned herein are exemplary. An alarm can be triggered as to notify the operator that such a condition has been met.

The tactile feedback system simulates this scenario by providing an ordered series of sensations such as containment, elasticity, dampening and finally saturation. If a perforation ensues, a sense of thickness will be appreciated as the catheter moves across the perforated tissue into a new virtual space. This series of tactile sensations is appropriate only when the catheter courses across the interatrial septum. These technologies can be incorporated into automatic and semi-automatic robotic systems.

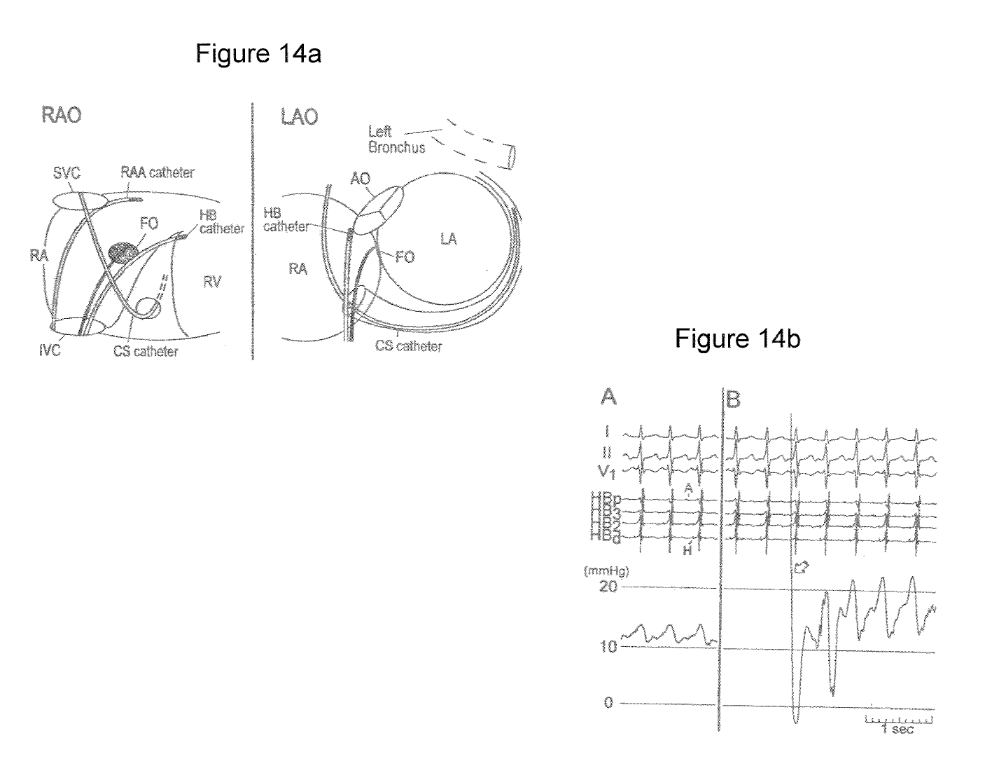

To further elaborate on the capability of the invention, we will describe its application to transeptal puncture techniques (FIGS. 14a and b). Those experienced in ablation techniques would be further assisted if they had an appreciation of contact with the inter-atrial septum (IAS) during transeptal puncture. During AF ablation, a catheter or catheters are manipulated from the RA across the IAS into the LA, (preferably across a patent foramen ovale present in approximately 20% of patients). This puncture technique requires careful manipulation of the catheter across the IAS as illustrated in FIGS. 14a and b. Such a catheter design is well known in the art and is comprised of a long vascular sheath similar to that manufactured by companies such as Biosense-Webster (Preface multipurpose) in conjunction with a Brockenbrough needle. In this mode of the invention an accelerometer is juxtaposed along the distal tip of a long vascular sheath and/or Brockenbrough needle.

In a preferred embodiment, the needle itself is constructed of material near the tip that has piezoelectric properties. The compliance and deformation properties of the catheter tip and/or needle will enable current generation from deformation and reduce risk of tissue damage. Referring to the bottom tracing of FIG. 14b, at time frame B, intracardiac pressure tracings are lost as the catheter crosses the IAS (Gonzales MD et al. Transeptal Left Heart Catheterization for Cardiac Ablation Procedures. J Interventional Cardiac Electrophysiology 5, 89-95, 2001). The amount of force generated on the IAS by the operator is essentially unknown with conventional systems as tactile appreciation of this force is limited secondary to the attenuation/damping characteristics of the catheter and surrounding vasculature/tissues.

In this mode of the invention, when the catheter crosses the IAS a characteristic abrupt change in the accelerometer derived signal will occur virtually simultaneously with the corresponding tactile stimulus from the catheter handle. Just prior to being transeptal the tactile feedback will, in order; generate a sense of enclosure (within RA), elasticity, damper effect and then a thickness sensation when opposed to and coursing through the IAS, respectively. Just prior to and after crossing the IAS a spring effect will be appreciated (note catheter fling in FIG. 14b).

Once within the left atrial chamber vibratory sensation is appreciated if the catheter is within the LAA during atrial fibrillation (10c and top 10d). This will have variable amplitude, vector and frequency. High frequency periodic movement (200-500 per minute), as known by those experienced in the art of game controllers will be appreciated. This movement will have an envelope with changes in level, gain, magnitude relative to atrial appendage motion. An attack and fade portion ramps from the attack level to the effect's overall magnitude over the attack time. As the catheter moves from a fibrillating appendage toward the pulmonary vein the level ramps from the effect's magnitude to a fade level over a fade time corresponding to the intra-cardiac forces imposed onto the catheter's distal portion. Thus, as the catheter tip moves toward a pulmonary vein (FIG. 10c), the vibratory amplitude will dampen. The periodic's waveform can be shaped (e.g. sinusoidal, triangular, sawtooth) relative to the changes in PzN current as a function of time. Spacing and bump width of each deflection provides texture type feedback. At optimal temporal resolution, this will simulate the texture of contacted tissues (e.g. ruggae of LAA (best appreciated in normal sinus rhythm), patent foramen ovale). TDI time graphs in FIG. 10c depict such changes in the periodic waveforms as the region of interest moves from a fibrillating LAA to pulmonary vein. Once within the pulmonary vein, intermittent constant force will be appreciated secondary to pulmonary venous inflow (bottom FIG. 10d) and the vibratory effect of the LAA disappears (top FIG. 10d).

The sample waveforms depicted in FIG. 10 are obtained by tissue and pulse wave Doppler transesophageal recordings from sample volumes in the specified regions of interest. Calibration of PzS or PzN current waveforms with TDI or other extrinsic modality will ensure accurate reproduction of tissue motion characteristics for data collection and for tactile feedback from the haptic handle of pacing leads and intra-cardiac catheters.

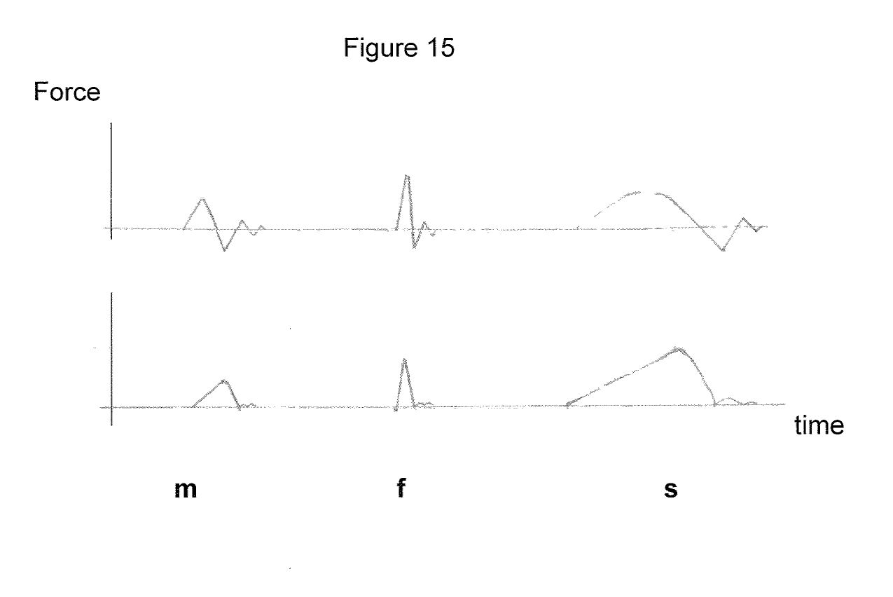

In FIG. 15, three examples of transeptal puncture are illustrated. The ordinate reflects coaxial (e.g. Z axis in FIG. 11) resistive force upon the catheter tip and is simulated by a force feedback system within the Haptic Handle. The abscissa is time. At s, the puncture is slow, at m it is intermediate, and at f it is fast. The images on the bottom reflect a rectified signal, though ideally, a to and fro motion is appreciated (top). A gradual puncture of the IAS is preferable as too abrupt of a puncture (point f* in FIG. 13) will increase risk (e.g. greater catheter force) and potentially result in uncontrolled motion that could lead to perforation of other anatomic structures such as the aorta or pericardial space resulting in tamponade and cardiovascular collapse. Thus, determination of catheter/needle location and applied force on the IAS with tactile sensation will vastly improve procedural success and ease such a technique. Optimally, the design of this technology is facilitated by validation with a plurality of conventional techniques such as simultaneous cross-correlation of data acquired with extrinsic navigational systems to that acquired with intrinsic PzS or PzN sensors and even intra-cardiac or transesophageal echocardiography.

Via the Haptic Handle, the operator will be able to detect when the catheter tip is intra-cavitary (sense of enclosure), juxtaposed to the IAS (thickness, stiffness, spring), within the LAA (periodic, texture), affected by blood flow at coronary sinus os (intermittent constant force), LA free wall (stiffness, spring, dampen), or near the mitral valve apparatus (vibration, constant force secondary to transmitral blood flow).

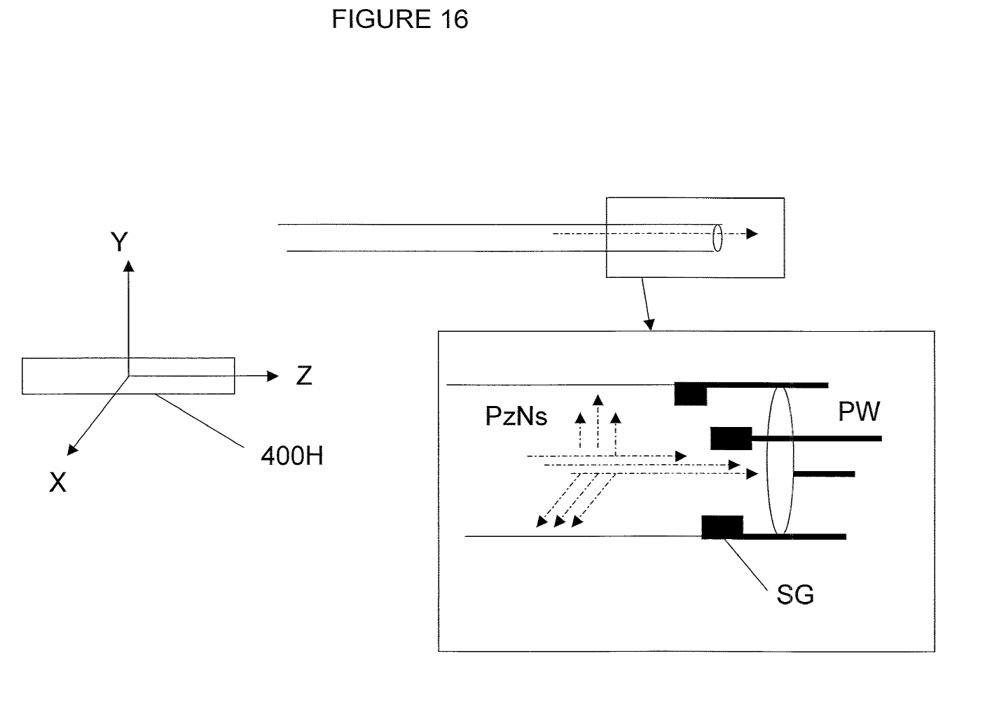

Similarly, the Haptic Handle will simulate the applied force upon an intravascular or intra-cavitary lead delivery or extraction system (e.g. Spectranetics) generating a sense of ramping resistance and friction. A finely calibrated sensor that has a low threshold for deformation and resultant current formation will be able to appreciate subtle changes in tissue character. In a preferred mode of the invention, characteristic changes in the sensor signal will distinguish the foramen ovale from the surrounding IAS, especially when a patent foramen ovale is present, as inter-atrial blood flow will cause some deformation of a catheter (soft biphasic constant force). Proprioceptive whiskers (PW) better enable recreation of intracardiac anatomy as discussed below (FIG. 16). The system is “taught” how to make such a differentiation by collection of a plurality of sensor signals while numerous patients undergo transeptal punctures under echocardiographic guidance (TEE or intra-cardiac echocardiography) and from comparisons with merged images acquired with CT angiography or other modality.

### Non-Piezoelectric Based Tactile Feedback

In an alternate embodiment, positional data from navigational systems is communicated back to the operator without a need for Pzs. The mechanical data can be obtained and used for diagnostic purposes but is also capable of being communicated to the operator via the tactile feedback system. Regional mechanical events at and about the location of the catheter tip in real-time is palpable in the catheter handle by simulating the motion of the catheter based on coordinates in three dimensions as defined by one or more navigational systems. Thus, the operator will appreciate characteristic anatomic mechanical properties such as the vibratory effects of the LAA during atrial fibrillation, the flow of blood from the pulmonary veins and degree of displacement and force generated by the contact of the catheter tip to and through the inter-atrial septum as described above.

It is expected that the temporal resolution of the navigational systems will be less than with PzN. Additionally, it is not standard of care to implant pacing leads using navigational systems and thus its application to technologies other than ablation of cardiac arrhythmias is more limited. The advantage of implementing a navigational system, however, is its ability to assist in recreation of a virtual intra-cardiac experience. Implementation of a plurality of signals including proprioceptive whiskers along with navigational technology and a haptic handle will not only serve to describe the location of the catheter but also will optimize the recreation of intra-cardiac geometry.

### Proprioceptive Whiskers

In an alternate embodiment, the degree or complete lack of tissue contact is palpable via a tactile feedback system that incorporates proprioceptive whiskers (PW) positioned about the most distal portion of the sheath. This will be optimal for guiding the most final and vital part of ablation procedures and supersede the need for more expensive imaging equipment (e.g. navigational systems, CT angiography). Four or more PW with directional strain gauges located at the basal portion of the PW provide the needed anatomic detail. By way of example, spring-steel wire or plastic polymer whiskers can be fitted with strain-gauges at the base for measurement of at least two orthogonal components of moment (Solomon J H, Hartmann M J. Robotic whiskers used to sense features. Nature 2006, vol 443, 525). Analysis of moments of each whisker using four radial contact points in space will enable extraction of information about anatomic shape. The PW can also detect properties of fluid flow (e.g. velocity, directionality, degree of laminar vs. turbulent flow) as long as whisker construct has adequate elasticity to be deformed from fluid flow. The strain gauges implemented are miniaturized as to fit within the catheter tip. Preferably, such miniaturization is accomplished using nanotechnology (e.g. parallel arranged nanospring transducers). The PW are oriented about the circumference of the catheter tip or sheath with strain gauges (SG) at their base and PzN may be located circumferentially alongside or more proximally (FIG. 16). From within the central lumen of the catheter the operator can manipulate a needle or inner catheter for ablation which can also have piezoelectric properties. PW technology, which is based on the physiology of whiskers found in marine animals (e.g. seal), has been shown to reconstruct three dimensional models of human facial features (Solomon J H, Hartmann M J. Robotic whiskers used to sense features. Nature 2006, vol 443, 525).

Initially, the PW system is “taught” anatomic landmarks and physiologic properties (e.g. left atrial appendage, pulmonary veins, intra-atrial septum, blood flow) by receiving concurrent input from extrinsic navigational systems, ultrasonic data, radiographic data and reconstructive programs during catheter or lead positioning attempts. Neural Networks and interpolation algorithms that have had anatomic data input from previously acquired images optimize the functioning of the system. These anatomic reconstructive programs can implement various imaging modalities (e.g. CT angiography, intra-cardiac echocardiography, MRI) with comparative data accessed, for example, using open connectivity.

### Nanogenerator and Biocompatible Electrical Interface

As described above, PzN, which in one embodiment is constructed of multiple gold-coated ZnO nanowires can be positioned about the tip of a intra-cardiac catheter/lead or woven into the fabric of carbon nanotube ribbons/scaffolds that are implanted along the cardiac surface (e.g. epicardial/pericardial). This and other PzN constructs generate voltage as a function of time and provide clinically useful information as described above. The durability of such a sensor is excellent over a range of frequencies and time and when multiple PzN are connected in parallel or series large amounts of generated piezoelectric potential is possible (e.g. 1-3 mV per cycle per PzN). A cardiac textile-fibre-based nanogenerator (NG) that is flexible, foldable and durable is expected to generate an output density of 20-80 mW/m.sup.2 (Qin Y, Wang X, Wang Z L. Microfibre-nanowire hybrid structure for energy scavenging. Nature. Vol 451, Feb. 14, 2008. 809-813). In one embodiment of the invention, the energy harvested by the NG is, in part or full, used for sensing, pacing and defibrillation. The mode of operation of the NG (e.g. monitoring PzN signals or harvesting energy) can be programmable or controlled by closed loop systems. Alternatively, the NG can switch modes (e.g. by multiplexing) and be multi-functional.

Referring to FIG. 17 we see a separate intra-cardiac lead (dashed black line in right ventricle, RV) with sensors (PzN) for generating monitoring current and an intra-pericardial PzN fabric or nanofelt (PzNf) for energy harvesting. PzNf can be manufactured using the techniques described above or in an alternate construct can consist of carbon nanotube fibers fabricated from single-wall carbon nanotubes using a particle coagulation spinning process. These “hair-like” conductive microwires (Young's modulus of the order of 1-3 GPa) are porous scaffolds that act as high-area electrodes consisting of permeable microfluidic conduits. Such a fabrication technique encourages the attachment, spreading and growth of mammalian cells within the construct ideal for a low impedance, biocompatible electrical interface (Dubin R A, Callegari G C, Kohn J, Neimark A V. Carbon Nanotube Fibers Are Compatible With Mammalian Cells and Neurons. IEEE Transactions on Nanobioscience, Vol. 7, No. 1, March 2008).

PzNf sensors provide clinically useful mechanical data to a CRM device at 5000 in form of a current waveform as a function of time. The data is available to be stored in the implanted CRM device (5000), a peripherally located processing center at 700 (e.g. device programmers, office based computers, extrinsic imaging modalities) and centrally located processing centers (e.g. academic research institutions, health care providers) at 1000. The current generated, Ipzn is conducted back to a capacitor (CAP) or battery (BATT) via either conventional conductors or carbon nanotube conductors (CNTC) as understood by those experienced in the art (Philip G. Collins and Phaedon Avouris (2000), Nanotubes for Electronics—Scientific American December 2000, 68-69).

Carbon nanotubes (CNT) have unique properties that render them useful for these applications. CNT's are extremely thin but have high electrical conductivity, very high tensile strength and are extremely flexible and elastic. Design of a lead using nanotechnology allows for multiple sensors/transducers to be incorporated without affecting structural properties, lead size and operative maneuverability.

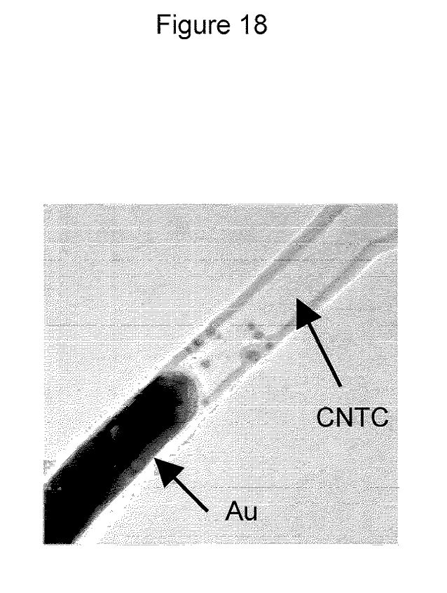

Hybrid conductors (HC) can serve to bridge conventional electrical conductors with CNTC (FIG. 17 and zoomed with electron microscopy in FIG. 18) within an endovascular or subcutaneous lead body and have been developed for other applications (Hybrid Structures Combine Strengths of Carbon Nanotubes and Nanowires. Rensselaer Polytechnic Institute. Applied Physics Letters 2007). The hybrid conductors combine conductors constructed using microfarbrication techniques (e.g. CNTC) and conventional conductors such as gold (FIG. 18) and will enable newer leads to be compatible with more conventional device hardware easing the CRM industry's transition into nanotechnology. The use of multiple CNTCs within a conventionally sized lead body enables bidirectional current flow for multiple applications (sensing myocardial depolarization, acquiring mechanical signals from multiple PzNs, delivery of pacing stimuli/defibrillation waveforms and NPIT, energy scavenging from a nanogenerator (e.g. PzNf)). In one embodiment of the invention, hybrid connectors (CNTC.fwdarw.CC) are present in the yolk of the lead and simplify the connections to device headers using conventional adapters (e.g. IS 1, IS 4).

At periodic intervals (e.g. hourly, weekly) charge is dumped from CAP to the BATT for storage (FIG. 17). CAP can also serve as an auxiliary power source in cases of primary battery failure. The current waveforms can be displayed for purposes of intra-cardiac monitoring and exhibited at 400. 400 can be a device programmer, computer terminal or tactile feedback system (400h). The tactile feedback system can be implemented at time of implant (HH in FIG. 17) to guide positioning of the intrinsic sensors, as well as, for initial data collection (double arrows between 400h, 400 and HH). Open communication (bidirectional wireless data transfer) to processing centers of the data at time of implant and chronically allows for calibration and standardization of an individual patient's clinical information as described above (e.g. cross-correlation to data acquired by one or both of extrinsic and intrinsic modalities). An individual's PzN sensor data (i.e. current), I.sub.data, flows via a conductor (e.g. CNTC) into a hybrid conductor into device 5000 in FIG. 17.

In one embodiment, the PzN sensors used for monitoring are woven into PzNf along specific vectors at various locations (e.g. AV groove, near valvular structures, myocardial wall segments) in order to monitor particular physiologic processes (e.g. ischemia, valvular function). Once processed this data is compared to other patients' data and assigned an appropriately scaled value in form of an index descriptive of cardiac performance (e.g. left ventricular torsion).

As described in the parent application, a cardiac performance index such as the torsion index, Ti, can be used to trigger activation of inotropic electrical stimulation (e.g. non-pharmacologic inotropic therapy or NPIT) at the appropriate times (i.e. when Ti is below a minimal threshold value of torsion, Tx) as illustrated in FIG. 17. NPIT can be delivered via a conventional conductor (CC in FIG. 17. NPIT current waveforms can be delivered to myocardium via PzNf, as well as, other electrode(s) and thus the PzNf can act as an active pace/sense (PS) electrode and nanogenerator. The therapeutic current waveforms (e.g. NPIT, pace, defibrillation) can be delivered to cardiac tissues via PzNf and/or different electrodes.

In one mode of the invention, the energy required for NPIT is in part of full, supplied by harvested energy from the nanogenerator, which can be stored within the battery, capacitor or alternate source, NG, as depicted in FIG. 17. In one mode, a large surface area PzNf is fabricated in an array of ribbon like fibers constructed of multiple ZnO nanowires positioned about the circumferential surface of the left ventricular free wall (e.g. intrapericardial, epicardial) as to increase the kinetic energy available for energy storage. Thus, the cardiac cycle dependent current waveforms that provide diagnostic data representative of torsional cardiac performance are used as a standardized metric of LV twist and harvest energy required for NPIT. Furthermore, the torsional physiologic data acquired by NG is incorporated into closed loop algorithms that trigger activation of NPIT at times where cardiac performance is compromised (as described in the parent application and FIG. 17 of this invention).

1-5. (canceled)

6. A haptic control system with haptic handle feedback, comprising:
a catheter adapted to be insertable into a vasculature of a patient, the catheter having a distal end that includes at least one microfabricated sensor;
a handle coupled to a proximal end of the catheter;
a haptic component associated with the handle that provides at least one of a vibratory and a motion sensation to an operator of the handle; and
a processor coupled to the handle, the processor configured to receive signals from the at least one microfabricated sensor and transform the signals into haptic representations of tissue motion by driving the haptic component, the haptic representations having an amplitude dependent on the magnitude of a real time parameter as sent by the at least one microfabricated sensor.

7. The haptic control system of claim 6, wherein the real time parameter is a cardiac parameter being selected from a group consisting on cardiac wall displacement magnitude, cardiac wall velocity, cardiac wall acceleration, blood flow, cardiac acoustic signals, cardiac rotation/torsion, cardiac tissue vibration, frequency of motion, cardiac arrhythmia, cardiac structure and cardiac geometry.

8. The haptic control system of claim 6, wherein the at least one microfabricated sensor comprises:
means for calibrating the at least one microfabricated sensor based on characterization of a data set representing cardiac parameters sensed from a multiplicity of patients.

9. The haptic control system of claim 7, wherein the at least one microfabricated sensor comprises:
means for calibrating the at least one microfabricated sensor based on characterization of a data set representing cardiac parameters sensed from a multiplicity of patients.

10. The haptic control system of claim 6, wherein the at least one microfabricated sensor comprises a piezoelectric sensor.

11. The haptic control system of claim 6, wherein the at least one microfabricated sensor comprises a nanosensor.

12. The haptic control system of claim 6, wherein the at least one microfabricated sensor comprises a proprioceptive whisker.
